(19) United States
(12) Patent Application Publication
(10) Pub. No.: US 2024/0358536 A1
(43) Pub. Date: Oct. 31, 2024

(54) Knee support brace and method of wearing same

(71) Applicant: Scott Geist, Hilton Head Island, SC (US)

(72) Inventor: Scott Geist, Hilton Head Island, SC (US)

(21) Appl. No.: 18/445,530

(22) Filed: Sep. 27, 2023

Related U.S. Application Data
(60) Provisional application No. 63/577,541, filed on Apr. 28, 2023.

(51) Int. Cl. A61F 5/01

(52) U.S. Cl. CPC A61F 5/0109

(57) ABSTRACT

The present invention discloses an improved knee support brace and a method of wearing the same. In a preferred embodiment, the knee support brace has a central brace panel foldable in configuration and formed having contoured vertical edges on opposite sides with an aperture centrally located therein adapted to be placed over the knee of the person wherein the aperture is located substantially in the middle of the panel substantially along the approximate vertical center line of the leg. The knee support brace also includes a pair of knee brace fastening means on each of the contoured vertical edges of the central brace panel and configured to secure the central brace panel in a wrapped position around the leg. The knee brace further includes a pair of tightening straps spaced apart above and below the central brace panel and connected thereto to tighten the central brace panel in wrapped position around the leg with at least one strap of the pair of straps having an elasticized section inserted intermediately along the strap to provide increased compression of the central brace panel in the wrapped position. A pair of ring loops are operatively connected to the central brace panel and disposed to engage respective ones of the pair of tightening straps so that the respective straps are routed about the central brace panel to secure the knee support brace upon the person with enhanced compression that prevents migration of the secured knee brace along the leg.

### CROSS-REFERENCE TO RELATED APPLICATION

This application claims the benefit of U.S. Provisional Application Ser. No. 63/577,541 filed Apr. 28, 2023 for a Knee Support Brace and Method of Wearing Same.

### FIELD OF THE INVENTION

The present disclosure relates to orthotic devices and in particular, to an improved knee support brace construction and method of wearing the same wherein the knee support brace includes a set of tightening straps adapted to overwrap an underlying support panel above and below the knee set in braced position, at least one of the tightening straps being constructed having an elasticized section inserted intermediately along its length to provide optimum compression of the brace support around the lower thigh and/or upper calf to prevent migration of the support brace along the leg when worn.

### BACKGROUND OF THE INVENTION

Knee support braces are very well known to obviate knee pain which is a very common problem faced by individuals during their lifetime. Treatments including medicines or physiotherapy are commonly used to address knee issues. In addition, knee support braces may be worn by individuals to assist in healing of knee injuries. Further, a knee brace supports kneecap, joints and muscles when they are weak, alleviates pain and enables a patient to move around during recovery from any injury.

There are several prior art patents covering knee support brace that provide rigid or flexible panels along the brace and brace adjustment means to try and achieve a proper fit for the person while at the same time providing firm and therapeutic support for the knee joint. However, it is extremely difficult to configure a knee brace with ideal fit without causing discomfort to the person wearing it. Moreover, most of the braces provide adjustment means that wear our very quickly or provide a very clumsy fit around the knee that can cause movement up or down along the leg reducing the desired support and causing additional discomfort.

One such recently granted patent, U.S. Pat. No. 10,709,593 issued Jul. 14, 2020, discloses a knee pain treatment apparatus that includes an upper member constructed to encircle a leg above a knee. The upper member includes an outer surface, an inner surface, an opening for exposing the front of the knee and a tensioning member integral with the upper member and depending downwardly therefrom. A patella biasing device is secured on the inner surface of the upper member and is arranged and constructed to border the opening. The apparatus further includes a flexible lower member arranged and constructed to encircle the leg below the knee and at least one tensioning member fastening system. The tensioning member fastening system is arranged and constructed for connecting the upper member and the lower member. When the upper member and lower member are mounted on the leg and connected, the tensioning fastening member may be stretched creating a downward biasing of the patella. While this and other prior art knee brace supports have served satisfactorily to support and stabilize the knee when fastened and fitted in place upon the wearer, those found in the prior art have not been found to adequately maintain a secure fit in relation to the knee size of a person and have evidenced migration of the knee brace from the fastened and fitted position reducing the support and stabilization intended.

In view of the above, there is a need for an improved knee support brace which can be worn for a long duration of time without discomfort and without migration from its fastened and fitted position upon the knee, which is easy to operate and may be suitably worn upon any individual regardless of the leg size.

### SUMMARY OF THE INVENTION

The inventive concepts presented herein are illustrated in a number of different embodiments, each showing one or more concepts, though it should be understood that, in general, the concepts are not mutually exclusive and may be used in combination even when not so illustrated.

Accordingly, in a primary aspect of an inventive embodiment, the subject invention provides an improved knee support brace comprising a central brace panel foldable in configuration and formed having similarly contoured edges running longitudinally along opposite sides with an aperture centrally located therein, the aperture being adapted to be placed over the knee of the person and located substantially in the middle of the panel substantially along the approximate vertical center line of the leg; a pair of knee brace fastening means on each of the contoured edges of the central brace panel and configured to secure the central brace panel in a wrapped position around the leg; a pair of tightening straps spaced apart above and below on the central brace panel and connected thereto at the base of each strap to tighten the central brace panel in wrapped position around the leg; and a pair of ring loops operatively connected to the central brace panel and disposed to engage a respective one of the tightening straps so that the respective strap is routed through and fastened about the central brace panel to secure the knee support brace upon the person with appropriate compression. At least one of the tightening straps, particularly the upper strap disposed above the aperture on the central brace panel for enwrapping the lower thigh, is constructed having an elasticized section insert intermediately along the strap to provide increased compression of the central brace panel about the thigh in the wrapped position and a comfortable fit upon the leg while preventing migration of the knee brace support upward or downward on the leg.

In a further embodiment, the central brace panel of the present knee support brace includes a first lateral pocket and a second lateral pocket disposed on each side of the central brace portion running longitudinally in a vertical direction thereupon for accommodating the pair of removable hinges, one on either side of the central brace panel. With the first and second lateral pockets thus vertically disposed, the pockets are intended to lie along and on each side of the approximate vertical center line of the wearer's knee when the knee support brace is in place with the hinges set in proper positions on either side of the knee.

In another embodiment, the present invention provides a method of wearing a knee support brace wherein the method includes the steps of adjusting a pair of members of a pair of removable hinges and inserting the hinges inside a pair of lateral pockets of a central brace panel of the knee support brace wherein each of the pair of lateral reinforcing means are vertically disposed and are intended to lie along and on each side of the approximate vertical center line of the wearer's leg, then placing the central brace panel of the knee support brace on a patella of the knee of the person and securing the knee brace by means of a pair of knee brace fastening means positioned upon the contoured edges of the central brace panel on opposite sides substantially parallel to the approximate vertical center line of the wearer's leg, before pulling a pair of tightening straps spaced above and below on the central brace panel and routed through respective ring loops operatively secured to the panel to tighten the central brace panel in wrapped position around the leg above and below the positioned knee, with at least one of the tightening straps having an elasticized section inserted intermediately along the strap to adjust compression of the central brace panel in the wrapped position and secure the fit of the knee support brace to knee, lower thigh and upper calf of the wearer with enhanced compression and without migration.

In yet another aspect of the subject invention, the pair of tightening straps are formed of a flexible length of a strap material having a smooth outer surface and hook and loop fasteners on the inner surface and further constructed having an elasticized section inserted along the length that creates increased compression when routed and wrapped about the central brace panel and provide a more secure fit of the knee brace on the wearer's leg.

To further clarify the advantages and features of the present invention, a more particular description of the invention will be rendered by reference to specific embodiments thereof, which are illustrated in the appended figures. It is appreciated that these figures depict only typical embodiments of the invention and are therefore not to be considered limiting of its scope.

### BRIEF DESCRIPTION OF THE DRAWINGS

The above contents of the present invention will become more readily apparent to those ordinarily skilled in the art after reviewing the following detailed description and accompanying drawings, in which:

FIG. 1 presents a layout view of the present knee support brace partially exploded to show a pair of hinges normally inserted in accordance with an embodiment of the invention;

FIG. 2 shows a overhead perspective view of the knee support brace partially secured about the knee with the lower tightening strap wrapped about the bottom of the central brace panel over the upper calf of the wearer and the upper tightening strap with its elasticized insert extended before encircling the back of the upper thigh to overwrap the top of the central brace panel and secure the knee brace upon the wearer in accordance with the present invention;

FIG. 3 shows an overhead perspective view of the present knee brace support with the upper tightening strap further being routed over the top of the central brace panel and through the ring loop to stretch the strap back around and over the panel with tension applied to the elasticized insert; and

FIG. 4 shows an overhead perspective view of the knee support brace fully wrapped in a closed assembly about the knee in accordance with an embodiment of the invention.

Further, skilled artisans will appreciate those elements in the figures are illustrated for simplicity and may not have been necessarily drawn to scale. Furthermore, in terms of the construction of the device, one or more standard components of the invention may have been generally depicted in favor of those more specific that are pertinent to understanding the embodiments of the present invention and so as not to obscure the figures with details that will be readily apparent to those of ordinary skill in the art having the benefit of the description herein.

### DETAILED DESCRIPTION OF THE INVENTION

Described herein are nonlimiting example embodiments of the present invention, which includes a knee support brace and a method of wearing the same.

The various embodiments including the example embodiments will now be described more fully with reference to the accompanying drawings, in which the various embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, the sizes of components may be exaggerated for clarity.

It will be understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

Spatially relative terms, such as “central brace panel”, “tightening strap”, or “elasticized insert” and the like, may be used herein for ease of description to describe one element or feature's relationship to another element(s) or feature(s) as illustrated in the figures. It will be understood that the spatially relative terms are intended to encompass different orientations of the structure in use or operation in addition to the orientation depicted in the figures.

The subject matter of various embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Generally, the various embodiments including the example embodiments relate to a knee support brace and a method of wearing the same.

Referring to the drawings, the following is a list of numerical references to elemental parts and components of the present back support brace and those associated structural elements shown throughout the drawings and employed in connection with the present invention:
100 knee support brace;
101 central brace panel;
102 top knee brace fastening means;
102a top knee brace fastening means at inner surface of the central brace panel;
102b top knee brace fastening means at outer surface of the central brace panel;
103 bottom knee brace fastening means;
103a bottom knee brace fastening means at inner surface of the central brace panel;
103b bottom knee brace fastening means at outer surface of the central brace panel;
104 top tightening strap;
105 bottom tightening strap;
106 elasticized insert section;
107 top ring loop of the pair of ring loops;
108 bottom ring loop of the pair of ring loops;
109,110 removable hinges;
109a,109b movable members of removable hinges;
110a,110b movable members of removable hinges;
109c,110c central hinge portion;
111,112 sleeves for accommodating the removable hinges;
113 vertical central line of wearers leg;
114 aperture as flattened cut-out; and
115,116 arcuate portions.

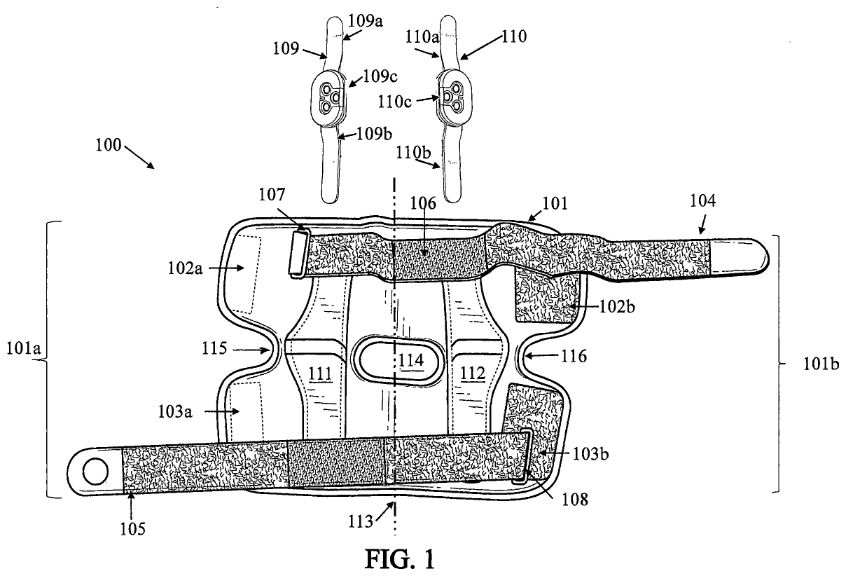

Referring initially to FIG. 1, a preferred embodiment of the knee support brace 100 is shown in a layout configuration partially exploded and viewed from the front or outer surface thereof. In accordance with the present invention, the knee support brace 100 includes a central brace panel 101 made of a soft and durable material and a pair of knee brace fastening means 102, 103 above and below on opposite side edges of the central brace panel to provide for the wrapped engagement of the opposite sides of the panel upon the knee. The knee support brace 100 further includes a pair of tightening straps 104, 105 each spaced apart and connected at one end thereof to the central brace panel 101, one tightening strap 104 connected along the upper edge of the central brace panel and the other strap 105 along the bottom edge of the panel with an associated pair of ring loops 107, 108 secured to the central brace panel at the juncture where each of the straps are connected. The pair of ring loops 107, 108 are positioned on exterior surface of the central brace panel 101 to properly route the respective tightening straps 104, 105 about the top and bottom of the panel and securely conform the panel to the knee, lower thigh and upper calf of the wearer. Both the tightening straps 104, 105 and the fastening means 102, 103 are made in their respective portions to engage and connect together using surfaces composed of hook and loop fasteners.

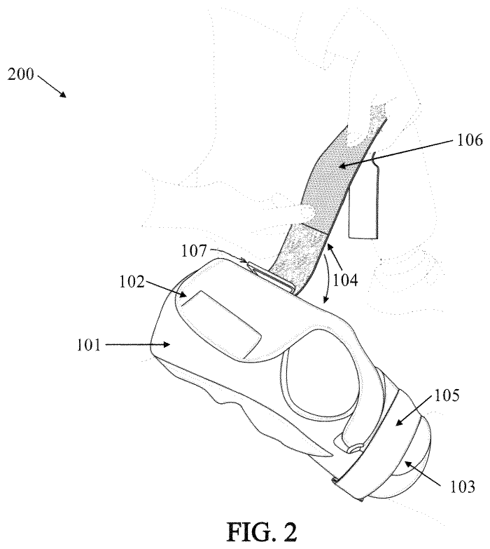

As shown and described more clearly below in references to FIGS. 2-4, the tightening straps 104, 105 are used to overwrap the central brace panel 101 about the knee and adjacent leg portions of the wearer to hold the present knee brace support 100 firmly in place, the upper strap 104 working about the lower thigh of the wearer while the lower strap 105 works about the upper calf. In accordance with the present invention, at least one of the tightening straps 104, 105, most beneficially the upper strap 104 as shown in FIG. 1, is constructed having an elasticized insert section 106 incorporated intermediately along the strap to provide a level of graduated tension to the tightening wrap of the strap and an increased yet suitable compression of the associated section of the central brace panel 101 when in the wrapped position about the knee.

The present knee support brace 100 further includes a pair of removable hinges 109, 110 configured to be placed inside pocket sleeves 111, 112 formed on either side of an aperture cutout 114 in the central brace panel 101 for providing lateral support to the knee. The central brace portion 101 of the knee support brace 100 includes a first lateral pocket sleeve 111 and a second lateral pocket sleeve 112 for accommodating the pair of removable hinges 109, 110. The first lateral pocket sleeve 111 and second lateral pocket sleeve 112 are vertically disposed and are intended to lie on either side of the aperture cutout 114 and alongside of the approximate vertical center line 113 of the wearer's leg. The aperture cutout 114 is located in the middle of the central brace panel 101 approximately midway between the first and second lateral pocket sleeves 111, 112 and serves as a patella opening for present knee support brace 100. To accommodate the patella, the aperture 114 is provided as a flattened cutout, which may be of different shapes including circular, oval, rectangle, or rhombus.

In its construction, the central brace panel 101 is made of a soft and pliable material and formed to be foldable along substantially vertical lines with contoured edges 101a, 101b running in a vertical direction along opposite sides of the central brace panel with the aperture 114 centrally located therein being adapted to be placed over the knee of the person. The aperture 114 is located substantially in the middle of the panel 101 substantially aligned with the projected vertical center line 113 of the wearer's leg. The various edges of the central brace panel 101 are stitched or glued to form a seam around the edges for enabling proper fit of the knee support brace 100 around the knee of the wearer.

In a preferred embodiment, each of the pair knee brace fastening means 102, 103 include a top fastening mean 102a, 103a and a bottom fastening means 102b, 103b that are positioned on each of the contoured vertical edges 101a, 101b of the central brace panel 101 and adapted to secure the central brace panel 101 in a wrapped position around the wearer's leg. The top fastening means 102a, 103a is positioned on the contoured vertical edge 101a and adapted to be fastened to the bottom fastening means 102b, 103b respectively positioned on contoured vertical edge 101b when the central brace panel is wrapped around the leg.

The tightening straps include an upper strap 104 and a bottom strap 105 that fasten above and below on the central brace panel 101 and are adapted to route in opposite directions. The combination of these tightening straps 104, 105 with elasticized insert section 106 overwrapping upon the central brace portion 101 provide improved fastening under tension when routed through the pair of ring loops 107, 108 enhancing the knee support provided by the knee support brace 100 upon its wrapped placement and reducing migration of the brace from its placement during usage. The pair of tightening straps 104, 105 are connected at one end to the central brace panel 101 together with a respective one of the ring loops 107, 108 and adapted to extend from and about the central brace panel 101 when place in wrapped position around the wearer's leg. The overall lengths of tightening straps 104, 105 including the normal unstretched length of the elasticized insert section 106 incorporated therein may vary but are preferably of sufficient lengths to overwrap the top and bottom of the central brace panel 101 and encircle the thigh or calf, as the case may be, to ensure an optimum and secure fit. The normal unstretched length of the elasticized insert section 106 will be increased by a dimensional or stretch factor upon tensioning the straps 104, 105 with that stretch factor being reflective of the added compression generated about the wrapped portion of the central brace panel 101. The width of the elasticized insert section 106 is generally the same as and uniform with the non-elasticized portion of the tightening straps 104, 105 with the width being made sufficient to apply the added compression upon the wrapped portions of the central brace panel in a secure and comfortable manner.

Each of the contoured vertical edges 101a, 101b of the central brace panel 101 includes an arcuate C-shaped middle portion 115, 116 shaped to form a substantially smooth close fit for the lower thigh and upper calf portions. The central hinge portions 109a, 109b, described further below, when positioned inside the respective pocket sleeves 111, 112 are substantially aligned with the arcuate portions 115, 116 and thereby provide a comfortable fit even when the person is moving. The pair of ring loops 107, 108 are disposed to engage a respective one of the pair of straps 104, 105 so that the respective strap may be routed around and fastened about the central brace panel 101 to secure the knee support brace 100 upon the wearer with suitable tension applied to the top and bottom of central brace panel that enhances its compression about the lower thigh and upper calf sections reducing migration of the position of the brace once in place.

In a further aspect of the present invention, the pair of removable hinges 109, 110 are configured to be placed inside the knee support brace 100 for providing lateral support to the knee, where the angle setting of each hinge is adjustable by the wearer before the hinge is inserted inside the respective pocket sleeves 111, 112 of the central brace panel 101 of the knee support brace 100. Each of the hinges 109, 110 includes a central hinge portion 109c, 110c having means to position the pair of movable arm members 109a, 109b and 110a, 110b at the appropriate angle in relation to the knee of the wearer. The central hinge portions 109c, 110c positioned inside the pocket sleeves 111, 112 rest in substantial alignment with the arcuate portions 115, 116 on each contoured vertical side edge of the central brace panel 101 thereby providing a smooth and comfortable fit even when the wearer is moving.

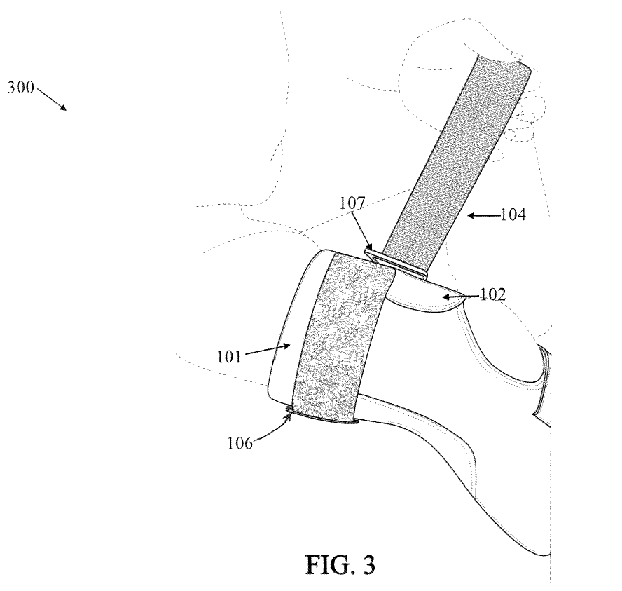

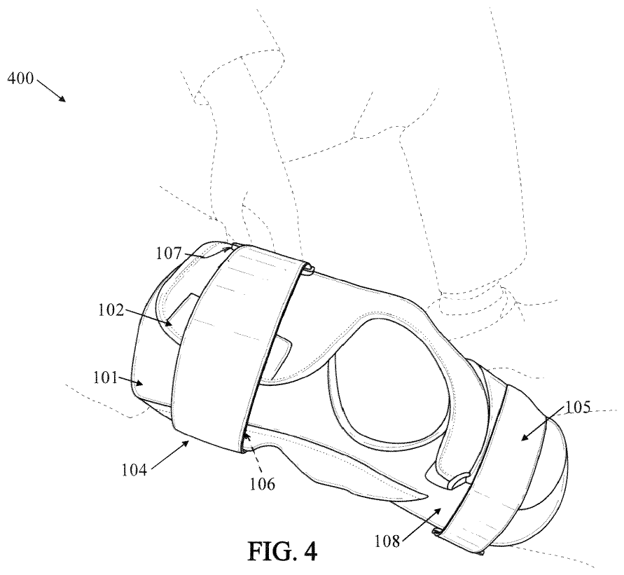

Referring now to FIGS. 2, 3 and 4 in conjunction with FIG. 1, perspective views of stages 200, 300, and 400 of the knee brace support 100 are shown in the method of effecting and wearing a fastened assembly of the knee brace support upon the leg of the wearer in accordance with the present invention. In reference to stage 200 in FIG. 2, with the knee support brace 100 holding the adjusted hinges 109, 110 inside the pocket sleeves 111, 112 on either side of the central aperture 114, the central brace panel 101 is placed over the leg and folded onto the patella of the knee so that the patella projects through the aperture with the contoured vertical edges of the central brace panel extending substantially parallel to the approximate vertical center line of the wearer's leg. In that position upon the leg, the central brace panel 101 is folded and wrapped about the leg with the upper portion of the panel being wrapped around the lower thigh and secured together with the fastener means 102 while the lower portion of the panel is wrapped around the upper calf and secured together with fastener means 103 before the bottom tightening strap 105 is routed and fastened about the lower portion of the panel. At this stage 200, the top tightening strap 104 incorporating the elasticized insert section 106 is extended from the central brace panel 101 and wrapped under and around the upper portion of the central brace panel to be routed through the ring loop 107 above on the central brace panel, as seen in stage 300 of FIG. 3, with the elasticized insert section being pulled longitudinally along with the top tightening strap and stretched under tension around the panel and over the lower thigh. Upon routing through ring loop 107 and fastening of the top tightening strap 104 to the exterior surface of the central brace panel 101, the tensioning of the elasticized insert section 106 in the top tightening strap results in enhanced compression of the upper portion of the central brace panel 101 upon the lower thigh, which compression may be adjusted by the wearer through easing or increasing the tension while fastening the top strap for a more comfortable but secure fit. Once routed back and then fastened in place, as seen in stage 400 of FIG. 4, the top tightening strap 104 with its elasticized insert 106 wrapped beneath and under tension is then fastened and secured in its proper position overtop the upper portion of the central brace panel 101 providing enhanced compression around the thigh in a suitable and comfortable amount sufficient to prevent movement of the panel and harmful migration of the knee support brace 100 from its proper therapeutic placement. It should be noted and understood that like the top tightening strap 104, the same construction of the bottom tightening strap 105 incorporating an elasticized insert section 106 may be used in accordance with the present invention to apply tensioned fastening of the lower central brace panel below the knee and about the upper calf to further enhance compression in that area and prevent migration of the present knee support brace 100.

While various elements of the knee support brace 100 including the tightening straps 104, 105 and fastening means 102, 103 are described and shown to be attached or connected through a hook and loop fastener system, such as VELCRO, it shall be apparent to a person skilled in the art that other connections may be used for securing these elements to the surfaces of the knee support brace within the scope of this invention.

Variations of the type, form, size, and material of any of central brace portion, top encircling strap, bottom encircling strap, hinges may be of any of those described previously and used in any of the method described.

Obviously, other embodiments and modifications of the present invention will readily come to those of ordinary skill in the art having the benefit of the teachings presented in the foregoing description and drawings. Alternate embodiments of different shapes and sizes, as well as substitution of known materials or those materials which may be developed at a future time to perform the same function as the present described embodiment are therefore considered to be part of the present invention. Furthermore, certain modifications to the described embodiment that serve to benefit its usage are within the scope of the present invention. Accordingly, it is understood that this invention is not limited to the particular embodiment described, but rather is intended to cover modifications within the spirit and scope of the present invention including the appended claims.

What is claimed is:

1. A knee support brace to be worn upon the leg of a person, comprising:
a central brace panel foldable in configuration and formed having contoured vertical edges on opposite sides thereof with an aperture cutout centrally located therein adapted to be placed over the knee substantially along the approximate vertical center line of the leg;
knee brace fastening means on each of the vertical edges of the central brace panel and configured to secure the central brace panel in a wrapped position around the leg;
a pair of straps spaced apart above and below the central brace panel and connected thereto to tighten the central brace panel in the wrapped position around the leg, at least one of the pair of straps incorporating an elasticized insert section intermediately along the strap to enhance compression of the central brace panel in the wrapped position; and
a pair of ring loops operatively connected to the central brace panel and disposed to engage a respective one of the pair of straps so that each respective strap is routed about the central brace panel and fastened to secure the knee support brace upon the person with enhanced compression and without migration.

2. The knee support brace of claim 1, wherein at least one of the pair of straps incorporating the elasticized insert section is disposed across an upper portion of the central brace panel for wrapping around the thigh of the wearer.

3. The knee support brace of claim 1, wherein at least one strap of the pair of straps incorporating the elasticized insert section is disposed across a lower portion of the central brace portion for wrapping around the calf of the wearer.

4. The knee support brace of claim 1, further comprising:
a pair of removable hinges configured for placement inside the central brace panel for providing lateral support to the knee.

5. The knee support brace of claim 4, wherein the central brace panel includes a first pocket sleeve and a second pocket sleeve for accommodating the pair of removable hinges wherein the first and second pocket sleeves are vertically disposed on the central bace panel for positioning on each side of the approximate vertical center line of the wearer's leg.

6. The knee support brace of claim 5, wherein the central brace panel further comprises a patella opening located approximately midway between the first and second pocket sleeve.

7. The knee support brace of claim 4, wherein each of the hinges includes a central hinge portion having means to position a pair of movable members on each side of the central hinge portion at the appropriate angle in relation to the knee of the person.

8. The knee support brace of claim 6, wherein the patella opening is located between the contoured vertical edges in alignment with arcuate portions inwardly formed along the contoured vertical edges and alongside the pocket sleeves so that the central hinge portions are positioned inside the sleeve pockets substantially parallel to the arcuate portion to provide a smooth fit of the brace even when the person is moving.

9. The knee support brace of claim 8, wherein the pair of straps are formed of flexible and elasticized material enabling secure fit of the knee brace on the leg of the person.

10. The knee support brace of claim 9, wherein hook and loop fasteners are positioned on exterior surfaces of the central brace panel to securely conform to the knee, thigh and calf of the person.

11. A method of wearing a knee support brace, the method comprising:
adjusting a pair of removable hinges and inserting the hinges inside a pair of lateral pocket sleeves on a central brace panel of the knee support brace to provide measured lateral support on each side of the knee;
placing the central brace panel of the knee support brace on a patella of the knee of the person and securing the knee brace by means of a pair of knee brace fastening means positioned on contoured vertical edges of the central brace panel;
routing a pair of straps spaced apart above and below on the central brace panel in opposite directions about the lower thigh and upper calf, wherein at least one of the straps is constructed having an elasticized section inserted intermediately along the length of strap;
tightening the straps while stretching the elasticized section about the central brace portion to apply enhanced compression upon the central brace panel and about the thigh and calf; and
fastening the straps along the length thereof to secure the knee support brace to knee, lower thigh and upper calf of the wearer with enhanced compression and without migration.

12. The method of claim 11, wherein the at least one strap of the pair of straps constructed having an elasticized section inserted intermediately along the length of strap is routed around the thigh of the wearer at a top brace portion above the central brace panel of the support brace.

13. The method of claim 11, wherein the at least one strap of the pair of straps constructed having an elasticized section inserted intermediately along the length of strap is routed around the calf of the wearer at a bottom brace portion below the central brace panel of the support brace.

14. The method of claim 11, wherein a patella opening is located approximately midway between the pair of lateral pocket sleeves.

15. The method of claim 14, wherein the hinge includes a central hinge portion having means to position a pair of movable members at the appropriate angle in relation to the knee of the person.

16. The method of claim 15, wherein hook and loop fasteners are positioned on exterior surfaces of the central brace panel to securely conform to the knee, lower thigh and upper calf of the person.
